SEQUENCE LISTING (11 sequences)

SEQ ID NO: 1 - protein, 118 aa, synthetic construct
QVQLQQSGPDLVKPGASVRISCKASGYTFTSYYIHWVKQRPGQGLEWIGWIYPGNVNTKYNEKFKGKATLTADKSSSTAYMQLSSLTSEDSAVYFCARRDYAINFDYWGQGTILTVSS

SEQ ID NO: 2 - protein, 112 aa, synthetic construct
DIMMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHFPWTFGGGTKLEIK

SEQ ID NO: 3 - protein, 15 aa, synthetic construct
GGGGSGGGGSGGGGS

SEQ ID NO: 4 - protein, 21 aa, synthetic construct
MALPVTALLLPLALLLHAARP

SEQ ID NO: 5 - protein, 45 aa, synthetic construct
TTTPAPRPPTPAPTIASQPLSLRPEACRPAAGGAVHTRGLDFACD

SEQ ID NO: 6 - protein, 27 aa, synthetic construct
MASPLTRFLSLNLLLLGESIILGSGEA

SEQ ID NO: 7 - protein, 42 aa, synthetic construct
KRGRKKLLYIFKQPFMRPVQTTQEEDGCSCRFPEEEEGGCEL

SEQ ID NO: 8 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 1106 aa, synthetic construct
MRLPGAMPALALKGELLLLSLLLLLEPQISQGLVVTPPGPELVLNVSSTFVLTCSGSAPVVWERMSQEPPQEMAKAQDGTFSSVLTLTNLTGLDTGEYFCTHNDSRGLETDERKRLYIFVPDPTVGFLPNDAEELFIFLTEITEITIPCRVTDPQLVVTLHEKKGDVALPVPYDHQRGFSGIFEDRSYICKTTIGDREVDSDAYYVYRLQVSSINVSVNAVQTVVRQGENITLMCIVIGNEVVNFEWTYPRKESGRLVEPVTDFLLDMPYHIRSILHIPSAELEDSGTYTCNVTESVNDHQDEKAINITVVESGYVRLLGEVGTLQFAELHRSRTLQVVFEAYPPPTVLWFKDNRTLGDSSAGEIALSTRNVSETRYVSELTLVRVKVAEAGHYTMRAFHEDAEVQLSFQLQINVPVRVLELSESHPDSGEQTVRCRGRGMPQPNIIWSACRDLKRCPRELPPTLLGNSSEEESQLETNVTYWEEEQEFEVVSTLRLQHVDRPLSVRCTLRNAVGQDTQEVIVVPHSLPFKVVVISAILALVVLTIISLIILIMLWQKKPRYEIRWKVIESVSSDGHEYIYVDPMQLPYDSTWELPRDQLVLGRTLGSGAFGQVVEATAHGLSHSQATMKVAVKMLKSTARSSEKQALMSELKIMSHLGPHLNVVNLLGACTKGGPIYIITEYCRYGDLVDYLHRNKHTFLQHHSDKRRPPSAELYSNALPVGLPLPSHVSLTGESDGGYMDMSKDESVDYVPMLDMKGDVKYADIESSNYMAPYDNYVPSAPERTCRATLINESPVLSYMDLVGFSYQVANGMEFLASKNCVHRDLAARNVLICEGKLVKICDFGLARDIMRDSNYISKGSTFLPLKWMAPESIFNSLYTTLSDVWSFGILLWEIFTLGGTPYPELPMNEQFYNAIKRGYRMAQPAHASDEIYEIMQKCWEEKFEIRPPFSQLVLLLERLLGEGYKKKYQQVDEEFLRSDHPAILRSQARLPGFHGLRSPLDTSSVLYTAVQPNEGDNDYIIPLPDPKPEVADEGPLEGSPSLASSTLNEVNTSSTISCDSPLEPQDEPEPEPQLELQVEPEPELEQLPDSGCPAPRAEAEDSFL

SEQ ID NO: 10 - protein, 468 aa, synthetic construct
LVVTPPGPELVLNVSSTFVLTCSGSAPVVWERMSQEPPQEMAKAQDGTFSSVLTLTNLTGLDTGEYFCTHNDSRGLETDERKRLYIFVPDPTVGFLPNDAEELFIFLTEITEITIPCRVTDPQLVVTLHEKKGDVALPVPYDHQRGFSGIFEDRSYICKTTIGDREVDSDAYYVYRLQVSSINVSVNAVQTVVRQGENITLMCIVIGNEVVNFEWTYPRKESGRLVEPVTDFLLDMPYHIRSILHIPSAELEDSGTYTCNVTESVNDHQDEKAINITVVESGYVRLLGEVGTLQFAELHRSRTLQVVFEAYPPPTVLWFKDNRTLGDSSAGEIALSTRNVSETRYVSELTLVRVKVAEAGHYTMRAFHEDAEVQLSFQLQINVPVRVLELSESHPDSGEQTVRCRGRGMPQPNIIWSACRDLKRCPRELPPTLLGNSSEEESQLETNVTYWEEEQEFEVVSTLRLQHV

SEQ ID NO: 11 - protein, 32 aa, synthetic construct
MRLPGAMPALALKGELLLLSLLLLLEPQISQG

(19) United States
(12) Patent Application Publication
(10) Pub. No.: US 2026/0061005 A1
(43) Pub. Date: Mar. 5, 2026

(54) SINGLE-CHAIN FRAGMENT VARIABLE TARGETING HUMAN PDGFR-BETA AND USE THEREOF IN CAR-T CELL IMMUNOTHERAPY

(71) Applicant: SHANDONG UNIVERSITY, Jinan (CN)

(72) Inventors: Fan YI, Jinan (CN); Wei TANG, Jinan (CN); Songbo ZHAO, Jinan (CN); Rongkun LI, Jinan (CN); Zhiyong LIU, Jinan (CN); Jiaying ZHANG, Jinan (CN)

(21) Appl. No.: 19/343,039

(22) Filed: Sep. 29, 2025

Related U.S. Application Data
(63) Continuation of application No. PCT/CN2025/083145, filed on Mar. 18, 2025.

(30) Foreign Application Priority Data
Jul. 16, 2024 (CN) 2024109527875

(51) Int. Cl. A61K 35/17; A61K 40/11; A61K 40/31; A61K 40/42; C07K 14/705; C07K 14/725; C07K 16/22; C12N 5/0783; C12N 15/86

(52) U.S. Cl. CPC A61K 35/17; A61K 40/11; A61K 40/31; A61K 40/4203; C07K 14/7051; C07K 14/70517; C07K 14/70578; C07K 16/22; C12N 5/0636; C12N 15/86; A61K 2239/13; A61K 2239/17; A61K 2239/21; C07K 2317/53; C07K 2317/622; C07K 2319/02; C07K 2319/03; C12N 2740/15043

(57) ABSTRACT

The present invention belongs to the technical fields of biomedicine and molecular biology, and particularly relates to a single-chain fragment variable (scFv) targeting human platelet-derived growth factor receptor (PDGFR)-β and use thereof in chimeric antigen receptor (CAR)-T cell immunotherapy. In the present invention, a scFv sequence targeting a human-derived PDGFRβ antigen is first obtained by immunizing a mouse, and then a second-generation CAR is constructed based on this, and additionally a CAR-T cell is obtained via lentivirus infection. The CAR-T cell can effectively kill a PDGFRβ antigen-positive 293T cell. The present invention provides a brand-new idea for eliminating PDGFRβ-positive cells to treat chronic kidney diseases, chronic liver diseases, cardiovascular diseases and various tumor diseases including various organ fibrosis, and has extremely attractive further development value and application prospects.

### REFERENCE TO SEQUENCE LISTING

The instant application contains a Sequence Listing in XML format as a file named “PCT2025007US-Sequence-Listing.xml”, created on Sep. 22, 2025, of 12,776 bytes in size, and which is hereby incorporated by reference in its entirety.

### CROSS-REFERENCE TO RELATED APPLICATIONS

The present invention claims priority to Chinese patent application No. 202410952787.5 filed with the China National Intellectual Property Administration on Jul. 16, 2024 and entitled “SINGLE-CHAIN FRAGMENT VARIABLE TARGETING HUMAN PDGFR-BETA AND USE THEREOF IN CAR-T CELL IMMUNOTHERAPY”, the entire content of which is incorporated by reference into the present invention and constitute a part of the present invention for all purposes.

### TECHNICAL FIELD

The present invention belongs to the technical fields of biomedicine and molecular biology, and particularly relates to a single-chain fragment variable (scFv) targeting human platelet-derived growth factor receptor β (PDGFR-beta or PDGFRβ) and use thereof in chimeric antigen receptor (CAR)-T cell immunotherapy.

### BACKGROUND

The information disclosed in the background art of the present invention is merely intended to enhance the understanding of the general background of the present invention, and is not necessarily to be construed as an admission or any form of implication that such information constitutes the prior art already known to those of ordinary skill in the art.

A kidney disease is a major global health problem that seriously threatens human health, and with the increasing severity of obesity, population aging and the like problems, the prevalence rate of a chronic kidney disease is up to over 10%. Studies have shown that it is predicted that by the end of this century, the chronic kidney disease will become the second leading cause of death in countries with longer life expectancy, and the chronic kidney disease will become the fifth most common cause of death worldwide. Renal fibrosis is a common pathological feature of chronic kidney diseases caused by various incentives. This pathological change is mainly caused by the activation of fibroblasts, pericytes, and myofibroblasts, etc. There is currently no effective and feasible treating means. Treating renal fibrosis and reversing renal function loss, etc. are current kidney problems that need to be addressed urgently.

Studies have shown that a PDGFRβ antigen is highly expressed on the surfaces of cells such as fibroblasts, pericytes and myofibroblasts. Eliminating cells such as fibroblasts, pericytes and myofibroblasts by targeting the PDGFRβ antigen is an effective and feasible approach. CAR-T cell immunotherapy has achieved great success in the treatment of blood cancers. In view of this, the CAR-T cell immunotherapy is also applicable to non-tumor diseases, such as autoimmune diseases, fibrosis, cardiovascular diseases, etc. Studies have shown that CAR-T cells targeting fibroblast activation protein (FAP) molecules can effectively inhibit myocardial fibrosis diseases, and CAR-T cells targeting urokinase-type plasminogen activator receptor (uPAR) molecules can alleviate liver fibrosis diseases by clearing senescent cells. However, the inventors have found that there is currently no scFv targeting human PDGFRβ antigen that can be used for constructing a CAR.

### SUMMARY

In view of the aforementioned prior art, an objective of the present invention is to provide a scFv targeting human PDGFRβ and use thereof in CAR-T cell immunotherapy. Specifically, a scFv targeting a human-derived PDGFRβ antigen is obtained by immunizing a mouse, and a second-generation CAR targeting the human PDGFRβ antigen is constructed. It is found that the CAR-T cells can effectively kill PDGFRβ antigen-positive cells, laying the foundation for CAR-T treatment of chronic kidney disease and other fibrosis-related diseases characterized by kidney damage. Based on the above research findings, the present disclosure is completed.

To achieve the above technical objective, the present disclosure provides the following technical solutions.

In a first aspect, a scFv targeting human PDGFRβ is provided, including a scFv heavy chain V_{H} and a scFv light chain V_{L}; where the scFv heavy chain V_{H} includes an amino acid sequence as shown in SEQ ID NO: 1 or a functional variant thereof; and the scFv light chain V_{L} includes an amino acid sequence as shown in SEQ ID NO: 2 or a functional variant thereof.

In the present invention, the term “functional variant” generally refers to an amino acid sequence that has substantially the same function as the scFv heavy chain or the scFv light chain (e.g., may having the properties of the single chain antibody or a chimeric antigen receptor), and has at least 85% (e.g., at least 85%, 90%, 91%, 92%, 93%, 94%, 95%, 96%, 97%, 98%, 99%, or at least 100%) sequence identity thereto. In certain specific embodiments, a variant of the amino acid sequence has substantially the same function as that of the amino acid sequence.

Further, the scFv heavy chain V_{H} and the scFv light chain V_{L} can be directly connected or connected through a linker; and the linker may be (G₄S)ₙ, where n is a positive integer, for example, 1, 2, 3, 4, 5 or 6, etc. In certain specific embodiments, the n is 3. Further, an amino acid sequence of (G₄S)₃ is as shown in SEQ ID NO: 3.

In a second aspect of the present invention, a chimeric antigen receptor (CAR) targeting human PDGFRβ is provided, which at least includes the aforementioned scFv targeting human PDGFRβ, and the scFv targeting human PDGFRβ is used as an antigen binding domain in the chimeric antigen receptor.

Further, the chimeric antigen receptor targeting human PDGFRβ is formed by connecting a signal peptide, an antigen binding domain, a hinge region, a transmembrane region, a costimulatory signaling domain and a signaling domain in series;

where the signal peptide can guide the antigen binding domain and the hinge region to transfer to a cell membrane surface. Any suitable signal peptide or combination of signal peptides can achieve the objective of the present invention. In one specific embodiment of the present invention, the signal peptide may be a cluster of differentiation 8 (CD8) signal peptide with an amino acid sequence as shown in SEQ ID NO: 4.

The hinge region may be a CD8 hinge region with an amino acid sequence as shown in SEQ ID NO: 5.

The transmembrane region may be a CD8 transmembrane region with an amino acid sequence as shown in SEQ ID NO: 6.

The costimulatory signaling domain may be a 4-1BB costimulatory signaling domain with an amino acid sequence as shown in SEQ ID NO: 7.

The signaling domain may be a cluster of differentiation 3ζ (CD3ζ) signaling domain with an amino acid sequence as shown in SEQ ID NO: 8.

Moreover, any peptide chain can be inserted as a spacer at a suitable position among the aforementioned antigen recognition region, hinge region, transmembrane region and intracellular signal region, and the peptide chain can be an oligopeptide or a polypeptide, which is not specifically limited here.

According to the present invention, the chimeric antigen receptor is formed by connecting a CD8 signal peptide, an antigen binding domain that binds to a human PDGFRβ antigen (V_{L}-(G₄S)₃-V_{H}), a CD8 hinge region, a CD8 transmembrane region, a 4-1BB costimulatory signaling domain, and a CD3ζ signaling domain in series.

In a third aspect of the present invention, an isolated nucleic acid molecule is provided, which encodes the aforementioned scFv or chimeric antigen receptor targeting human PDGFRβ.

In a fourth aspect of the present invention, a vector including the aforementioned nucleic acid molecule is provided.

According to the present invention, the vector is a viral vector, which may be a retroviral vector or a lentiviral vector; further preferably a lentiviral vector. The vector is a recombinant viral vector expressing the aforementioned scFv or chimeric antigen receptor obtained by inserting the nucleic acid molecule encoding the aforementioned scFv or chimeric antigen receptor into a virus.

In a fifth aspect of the present invention, an immunocompetent cell is provided, which expresses any of the aforementioned single-chain antibodies or chimeric antigen receptors; or, includes a nucleic acid molecule encoding any of the aforementioned single-chain antibodies or chimeric antigen receptors; preferably, the immunocompetent cell is selected from: a T cell, a natural killer (NK) cell, a monocyte, a macrophage, a dendritic cell or a mast cell; where the T cell is preferred, and further, when it expresses a chimeric antibody receptor or includes a nucleic acid molecule encoding a chimeric antigen receptor, the immunocompetent cell is a CAR-T cell.

In a sixth aspect of the present invention, a method for preparing the immunocompetent cell (particularly a CAR-T cell) is provided, including: infecting a T cell with a lentivirus; where the lentivirus is obtained by transfecting a recombinant lentiviral vector into a lentiviral packaging cell, followed by cell culture; and the recombinant lentiviral vector is prepared by inserting a nucleic acid molecule encoding the aforementioned chimeric antigen receptor into a lentiviral vector.

In a seventh aspect of the present invention, a pharmaceutical composition is provided, which includes a therapeutically effective amount of one or a combination of: any of the aforementioned single-chain antibodies; any of the aforementioned chimeric antigen receptors; any of the aforementioned immunocompetent cells; any of the aforementioned nucleic acid molecules, vectors, or a product prepared by any of the aforementioned methods, and a pharmaceutically acceptable carrier.

In an eighth aspect of the present invention, use of any of the aforementioned single-chain antibodies; any of the aforementioned chimeric antigen receptors; any of the aforementioned immunocompetent cells; any of the aforementioned nucleic acid molecules, vectors, or a product or pharmaceutical composition prepared by any of the aforementioned methods in preparation of a drug for preventing and/or treating a disease mediated by high PDGFRβ expression, is provided.

Specifically, the present invention has confirmed through research that the PDGFRβ CAR-T cell of the present invention has a strong killing ability against a platelet-derived growth factor receptor β⁺ (PDGFRβ⁺) cell in vitro. Therefore, the disease mediated by high PDGFRβ expression includes, but is not limited to a kidney disease, a liver disease, a cardiovascular disease and a tumor disease, especially a fibrosis-mediated chronic kidney disease, liver disease, cardiovascular disease and tumor disease.

In a ninth aspect of the present invention, a method for preventing and/or treating a disease mediated by high PDGFRβ expression is provided, including administering to a subject a therapeutically effective amount of any of the aforementioned single-chain antibodies; any of the aforementioned chimeric antigen receptors; any of the aforementioned immunocompetent cells; any of the aforementioned nucleic acid molecules, vectors, or a product or pharmaceutical composition prepared by any of the aforementioned methods.

In a tenth aspect of the present invention, a kit is provided, including any of the aforementioned single-chain antibodies; any of the aforementioned chimeric antigen receptors; any of the aforementioned immunocompetent cells; any of the aforementioned nucleic acid molecules, vectors, or a product or pharmaceutical composition prepared by any of the aforementioned methods.

In an eleventh aspect of the present invention, use of any of the aforementioned single-chain antibodies; any of the aforementioned chimeric antigen receptors; any of the aforementioned immunocompetent cells; any of the aforementioned nucleic acid molecules, vectors, or a product, pharmaceutical composition or kit prepared by any of the aforementioned methods in detecting PDGFRβ expression or in preparation of a product for detecting PDGFRβ expression, is provided.

Specifically, qualitative or quantitative detection of PDGFRβ expression in a subject sample can be used for screening, (assisted) diagnosis, or prediction of the progression of a disease mediated by high PDGFRβ expression.

### Beneficial Technical Effects of One or More of the Above Technical Solutions

The aforementioned technical solutions first obtain the scFv sequence targeting the human PDGFRβ antigen by immunizing a mouse, and then constructs the second-generation CAR on this basis. Additionally, the CAR-T cell is obtained by lentiviral infection. The CAR-T cell can effectively kill the PDGFRβ antigen-positive 293T cell.

The aforementioned technical solution provides a brand-new idea for eliminating PDGFRβ-positive cells to treat chronic kidney diseases, liver diseases, cardiovascular diseases and various tumor diseases including various organ fibrosis, and has extremely attractive further development value and application prospects.

### BRIEF DESCRIPTION OF THE DRAWINGS

The accompanying drawings of the specification which form a part of the present invention are used for providing further understanding of the present invention. The illustrative embodiments of the present invention and the description thereof are used for explaining the present invention, and do not constitute improper limitation of the present invention.

FIG. 1A shows the detection of a PDGFRβ extracellular region protein before purification in an embodiment of the present invention, and respective bands are respectively 1: uninduced bacteria, 2-4: induced bacteria, 5: a precipitate after induction, 6: a supernatant after induction, and M: a marker.

FIG. 1B shows the detection of the PDGFRβ extracellular region protein after purification in an embodiment of the present invention, and respective bands are respectively 1: a purified protein, M: a marker, and BSA: bovine serum albumin.

FIG. 2 shows the process of mouse immunization and acquisition of monoclonal antibody cell strains in an embodiment of the present invention.

FIG. 3A shows the PDGFRβ CAR structure in an embodiment of the present invention.

FIG. 3B shows a lentiviral vector used for constructing a CAR in an embodiment of the present invention.

FIG. 3C shows the detection of a positive rate of a PDGFRβ CAR-T cell in an embodiment of the present invention.

FIG. 4A is a flow cytometry diagram of construction of a 293T-PDGFRβ cell in an embodiment of the present invention.

FIG. 4B shows a PDGFRβ CAR-T cell killing curve in an embodiment of the present invention.

### DETAILED DESCRIPTION

It should be pointed out that the following detailed descriptions are all exemplary and are intended to provide further illustration of the present disclosure. Unless otherwise specified, all technical terms and scientific terms used herein have the same meanings as commonly understood by those of ordinary skill in the art to which the present disclosure belongs.

It should be noted that the terms used herein are only for describing specific embodiments and are not intended to limit the exemplary embodiments according to the present invention. As used herein, unless otherwise expressly stated in the context, singular forms are also intended to include plural forms. In addition, it should also be understood that when the terms “include” and/or “comprise” are used in the specification, the terms indicate existence of features, steps, operations, devices, components, and/or combinations thereof.

The present invention will now be further described in connection with specific examples. The following examples are only intended to explain the present invention, rather than limiting its contents. If the specific experimental conditions are not specified in the examples, they are usually according to conventional conditions or the conditions recommended by reagent companies; and unless otherwise specified, the reagents, consumables and the like used in the following examples all can be obtained from commercial channels.

In the present invention, the term “specifically bind” refers to that an antigen binding molecule (e.g., an antibody) typically specifically binds to an antigen and substantially the same antigen with high affinity, but does not bind to unrelated antigens with high affinity. The affinity is generally reflected by an equilibrium dissociation constant (K_{D}), where a lower K_{D} indicates a higher affinity.

In the present invention, the term “antibody” is used in the broadest sense, and refers to a polypeptide or polypeptide combination that contains sufficient sequences from an immunoglobulin heavy chain variable region and/or sufficient sequences from an immunoglobulin light chain variable region to specifically bind to an antigen. The “antibody” herein encompasses various forms and various structures, as long as they exhibit the desired antigen binding activity.

In the present invention, the term “monoclonal antibody” refers to an antibody molecule preparation having a single molecular composition. The monoclonal antibody displays a single binding specificity and affinity for a particular epitope.

In the present invention, the term “antibody” can be derived from any animal, including but not limited to human and non-human animals, where the non-human animals can be selected from primates, mammals, rodents and vertebrates, such as camelids, llamas, ostriches, alpacas, sheep, rabbits, mice, rats or Chondrichthyes (e.g. sharks).

In the present invention, the term “antibody” includes, but is not limited to, a monoclonal antibody, a polyclonal antibody, a monospecific antibody, a multispecific antibody (e.g., a bispecific antibody), a monovalent antibody, a multivalent antibody, an intact antibody, an antigen-binding fragment, a naked antibody, a conjugated antibody, a humanized antibody, or a fully human antibody.

In the present invention, the term “humanized antibody” refers to a non-human-derived antibody which has been genetically engineered and of which the amino acid sequence has been modified to increase the sequence homology with a human-derived antibody. Generally speaking, all or part of a complementarity determining region (CDR) of a humanized antibody is derived from a non-human-derived antibody (donor antibody), and all or part of a non-CDR region (e.g., variable framework region (FR) and/or constant region) is derived from a human-derived immunoglobulin (recipient antibody). The humanized antibody generally retains or partially retains the desired properties of the donor antibody, including but not limited to antigen specificity, affinity, reactivity, ability to increase immune cell activity, ability to enhance immune response, etc.

In the present invention, the terms “identity” and “consistency” are interchangeable and are obtained by calculation in the following manner: to determine the percentage of “identity” between two amino acid sequences or two nucleic acid sequences, the sequences are aligned for optimal comparison purposes (for example, gaps can be introduced into one or both of the first and second amino acid sequences or nucleic acid sequences for optimal alignment, or non-homologous sequences can be discarded for comparison purposes). The amino acid residues or nucleotides at corresponding amino acid positions or nucleotide positions are then compared. When a position in the first sequence is occupied by the same amino acid residue or nucleotide at the corresponding position in the second sequence, then the molecules are identical at that position.

In the present invention, the term “immunocompetent cell” refers to a cell that is responsible for an immune function in an organism. Examples of immunocompetent cells may include for example lymphocytes such as T cells, natural killer cells (NK cells), B cells, etc.; antigen-presenting cells such as monocytes, macrophages, dendritic cells, etc.; and granulocytes such as neutrophilic granulocytes, eosinophilic granulocytes, basophilic granulocytes, mast cells, etc. Specifically, preferred examples include T cells or NK cells derived from mammals such as human, dogs, cats, pigs, and mice, etc., and preferably T cells or NK cells derived from human. Furthermore, T cells can be isolated and purified from body fluids such as blood and bone marrow fluid, tissues such as the spleen, thymus, and lymph nodes, or immunocompetent cells infiltrating cancerous tissues such as primary tumors, metastatic tumors, and cancerous ascites. T cells made from embryonic stem cells (ESCs) and induced pluripotent stem cells (iPSCs) can also be utilized. It should be noted that, the source of the immunocompetent cells and the object to be administered may be the same or different. In turn, when the object to be administered is human, the immunocompetent cells may be autologous cells collected from a patient herself or himself as the object to be administered, or allogeneic cells collected from others. That is, the donor and the recipient may or may not be identical, but are preferably identical.

In the present invention, the term “pharmaceutically acceptable carrier” includes any and all solvents, dispersion media, coatings, antibacterial and antifungal agents, isotonic agents and absorption delaying agents, and the like that are physiologically compatible. In general, the nature of the carrier will depend on the employed particular administration mode. For example, a parenteral formulation general contains an injectable fluid as a vehicle, which contains a pharmaceutically and physiologically acceptable fluid such as water, physiological saline, a balanced salt solution, an aqueous glucose solution, glycerol, and the like. For solid compositions (e.g., powder, pill, tablet, or capsule forms), conventional non-toxic solid carriers can include, for example, pharmaceutical-grade mannitol, lactose, starch, or magnesium stearate. In addition to biologically neutral carriers, the pharmaceutical composition to be dosed may further contain minor amounts of non-toxic auxiliary substances, such as wetting agents or emulsifying agents, preservatives, and pH buffering agents, etc., for example sodium acetate or sorbitan monolaurate.

In the present invention, the term “therapeutically effective amount” refers to an amount that effectively achieves a desired therapeutic or preventive result in a necessary dose and for a necessary time period. A therapeutically effective amount of an agent, for example, eliminates, reduces, delays, minimizes, or prevents an adverse effect of a disease.

The terms “treating and/or preventing” refer to attempts to alter the natural course of a disease in a treated individual, and can be clinical intervention implemented either for prevention or during the course of clinical pathology. Desirable effects of treatment include, but are not limited to, preventing the occurrence or recurrence of a disease, alleviating a symptom, reducing any direct or indirect pathological consequences of a disease, preventing metastasis, slowing the rate of disease progression, improving or alleviating a disease state, and eliminating or improving prognosis. In some embodiments, the antibodies of the present invention are used for delaying the development of a disease or delay the progression of a condition.

In the present invention, an “individual” or “subject” is preferably a mammal. The mammal includes human and a non-human mammal, and the non-human mammal includes a mouse, rat, guinea pig, cow, sheep, cat, dog, horse, monkey, oran-gutan, etc., where the individual or subject is preferably human.

Specifically, the cell designed in the present invention is a human-derived T cell.

The human-derived T cell is derived from peripheral blood, and activated by stimulation with CD3/cluster of differentiation 28 (CD28) magnetic beads.

The CAR-T cell used in the present invention is constructed by lentiviral infection.

The lentiviral vector is pCDH-EF1-MCS-T2A-copGFP, and a relevant lentivirus is obtained by packaging in a second-generation lentiviral packaging manner, using auxiliary plasmids PSPAX2 and PM2G, with the multiplicity of infection (MOI)=20.

The CAR-T cell targeting human PDGFRβ of the present invention can well kill a 293T cell expressing PDGFRβ⁺ in vitro.

The present invention is further illustrated by examples hereafter, but the examples do not constitute a limitation of the present invention. It should be understood that, the following examples are only intended to illustrate the present invention, rather than limiting the scope of the present invention. The test methods in the following examples which are not specified with specific conditions are generally carried out according to conventional conditions.

### EXAMPLES

### Method

### Mouse Immunization Process

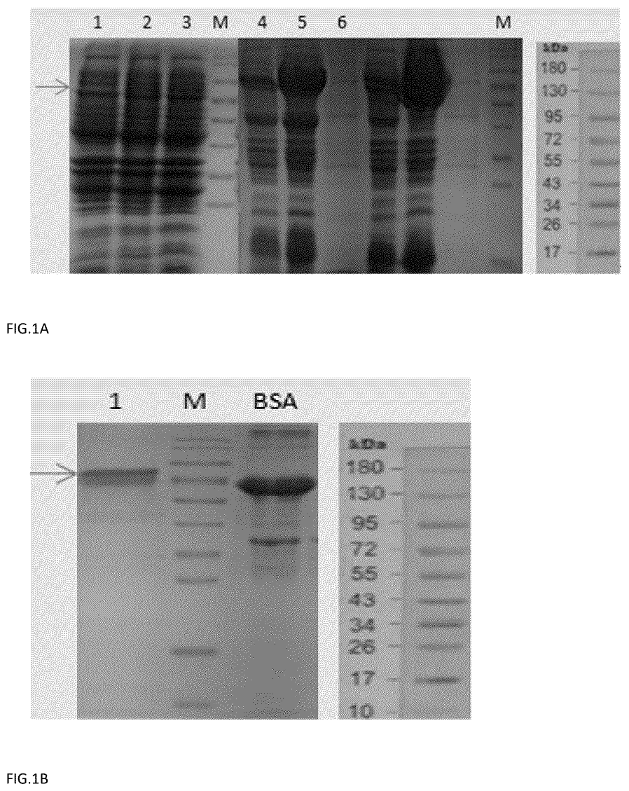

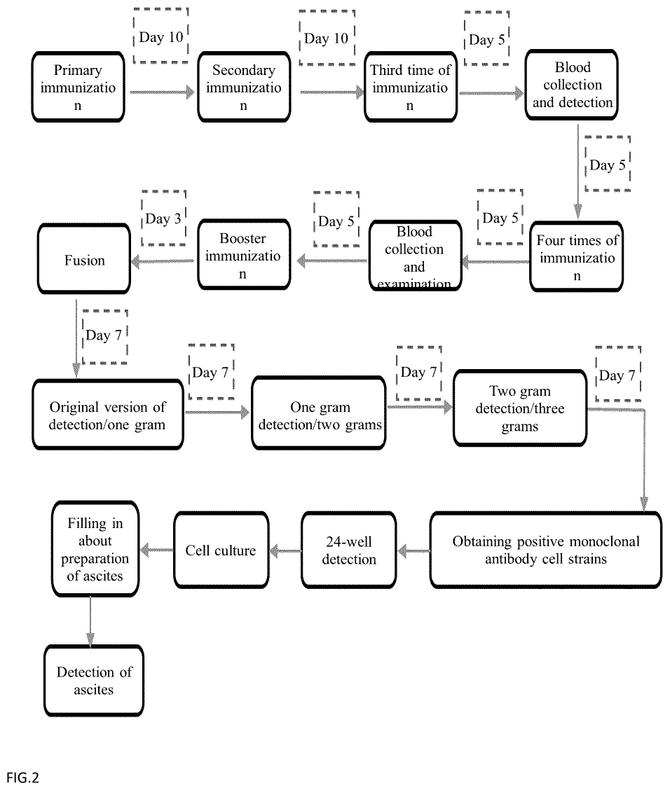

An amino acid sequence of a human PDGFRβ antigen is as shown in SEQ ID NO: 9. Gene synthesis design was conducted by constructing an extracellular region sequence of the human PDGFRβ antigen (SEQ ID NO: 10) onto a pcDNA3.0 vector, and adding a membrane localization signal peptide (SEQ ID NO: 11) in front of it. 2 μL of a plasmid was added into 50 μL of BL21 competent bacteria, and subjected to ice bath for 30 min. The bacteria were heat shocked at 42° C. for 90 s, quickly placed into ice for 5 min, and added with 500 μL of a Luria-Bertani (LB) culture medium. The bacteria were shaken at 37° C. and 220 rpm for 1 h, coated onto an LB plate containing ampicillin resistance, and cultured in an inverted manner at 37° C. overnight. Single colonies were picked from the plate and inoculated into a test tube containing 5 mL of an LB culture medium containing ampicillin resistance. The mixture was shaken at 37° C. and 200 rpm until OD₀₀ of the culture solution was 0.6-0.8. The culture solution was added with isopropyl β-D-1-thiogalactopyranoside (IPTG) to a final concentration of 0.1 mM, and meanwhile a culture solution without addition of the IPTG was set as a control. The culture solution was shaken at 16° C. and 200 rpm for 16 h to induce the expression of a fusion protein. 1 mL of the culture was taken out, and centrifuged at 12,000 g for 10 min at room temperature. The supernatant was discarded, and the bacterial pellet was resuspended with 50 μL of a 1× phosphate-buffered saline (PBS) buffer solution and added with 25 μL of a 3× Loading Buffer. The solution was analyzed by 12% sodium dodecyl sulfate-polyacrylamide gel electrophoresis (SDS-PAGE), which showed that the fusion protein was expressed at approximately 80 kDa, mainly in the form of inclusion bodies. A constructed band was found by protein expression identification technology (FIG. 1A), and then a relatively pure PDGFRβ antigen extracellular region protein was obtained by protein purification technology (FIG. 1B). The immunization process was as shown in FIG. 2. 4-week-old Balb/c mice were immunized with the PDGFRβ extracellular region protein purified from eukaryotic cells via tail vein reinfusion, with the immunization dose of 100 μg per mouse per time, and a total of four times of immunization were performed. Fusion was performed 13 days after the end of the immunization.

Screening of High Affinity PDGFRβ scFv

Blood collection and detection: immunization was performed according to a mature immunization process, and titer determination and the like indicator detections were completed. In this example, the mice corresponding to the effectively obtained optimal cell strain and antibody results, and related data are summarized and summarized. As shown in Table 1, the data of multiple rounds of blood collection and detection showed that the antibody titer of mouse 2 # was the highest, so the mouse 2 # was selected for hybridoma fusion.

Fusion and cloning: SP2/0 mouse myeloma cells were utilized for cell fusion with spleen cells of the preferred mouse (2 #). After the fusion, a batch of hybridoma cells that met the experimental requirements were obtained through culture, observation, detection and positive and negative control tests, and they were cultured continually and selected.

Strain identification and sequencing of the mouse 2 #: after fusion, primary cloning by limited dilution, and supernatant detection of the mice, a total of 31 cell strains of 14 types that met the project requirements, were obtained. By comparison, a hybridoma with higher titer and optimal affinity was sequenced to obtain the full-length PDGFRβ antibody sequence. The antibody heavy chain/light chain variable region genes were obtained by polymerase chain reaction (PCR), and thus the PDGFRβ scFv was obtained.

TABLE 1
Results of blood collection and detection

| | |
|---|---|
| Project Name | Human PDGFRβ antibody acquisition |
| Immunogen | PDGFRβ antigen extracellular region |
| Coated antigen | PDGFRβ antigen extracellular region |
| Coating fluid | 0.05M CB pH 9.6 |
| Blocking | Conventional blocking solution |
| solution | |
| Antibody diluent | 0.02M PBS |
| Sample addition | 100 μl/well |
| manner | |
| Reaction | 30 min-30 min-15 min, 37° C. |

| Number of | Mouse serial | Antigen dilution | | | |
|---|---|---|---|---|---|
| blood collection | number | concentration | Antibody titer | | OD value |
| First time of | 1# | 1 μg/ml | 1.7 | w | 1.2 |
| blood collection | 2# | 1 μg/ml | 4.1 | w | 2.5 |
| and detection | 3# | 1 μg/ml | 2.7 | w | 2.4 |
| | 4# | 1 μg/ml | 1 | k | 1.5 |
| | 5# | 1 μg/ml | 3 | k | 1.9 |
| Second time of | 1# | 1 μg/ml | 3.1 | w | 1.6 |
| blood collection | 2# | 1 μg/ml | 9.1 | w | 2.7 |
| and detection | 3# | 1 μg/ml | 4.3 | w | 1.6 |
| | 4# | 1 μg/ml | 2.7 | w | 1.8 |
| | 5# | 1 μg/ml | 8.1 | w | 1.7 |
| Third time of | 1# | 1 μg/ml | 12.1 | w | 1.5 |
| blood collection | 2# | 1 μg/ml | 24.3 | w | 1.4 |
| and detection | 4# | 1 μg/ml | 8.1 | w | 1.6 |
| | 5# | 1 μg/ml | 18.3 | w | 1.3 |

### CAR Structure

The sequence in the CAR of this example was CD8 signal peptide-VL-(G₄S)₃-VH-CD8 hinge region-CD8 transmembrane region-4-11BB-CD. The sequence was synthesized by Qingke Biotechnology Co., Ltd., and constructed onto a designated vector pCDH-EF1-MCS-T2A-copGFP (FIG. 31B), with the front and back enzymatic cleavage sites being XbaI and BamHI, respectively. After sequencing and alignment, the sequences were found to be consistent. The sequence was packaged into a lentivirus using a second-generation viral packaging system (with the packaging plasmids of PSPAX2 and PM2G).

### Feeder Cell Preparation

BALB/C mice were sacrificed by neck dislocating, and disinfected by soaking in 75% alcohol for 5-10 min. A small incision was made in the abdomen of the mouse with surgical scissors, and the skin was peeled off to expose the abdominal cavity. The peritoneal membrane was clamped and lifted up with hemostatic forceps (without clamping the abdominal organs), 4 ml-5 ml of a dulbecco's modified eagle medium (DMEM) solution was injected into the abdominal cavity with a syringe. The legs of the mouse were clamped with forceps and the mouse was shaken back and forth for about 1 min, the peritoneal fluid was drawn back with the same syringe, and added into a centrifuge tube. This process was repeated for 2-3 times. The fluid was centrifuged at 1,000 rpm for 10 min. The supernatant was discarded, and the cells were prepared into a suspension with a HAT selection medium.

### Preparation of Spleen Cells and Myeloma Cells

The mice were sacrificed by neck dislocating, and disinfected by soaking in 75% alcohol for 5 min-10 min. The mice were taken out for dissection and the spleens were removed under sterile conditions. The spleen was rinsed in 5 ml of a DMEM solution to wash off the red blood cells on the spleen. DMEM was pipetted with a 5 ml syringe to pipette the spleen up and down to prepare a spleen cell suspension, and the suspension was transferred into a 50 ml centrifuge tube. This process was repeated for several times until the spleen became transparent. The culture medium of SP2/0 cultured in advance was discarded, and the SP2/0 was pipetted off with new DMEM preheated at 37° C. in advance, and transferred into the centrifuge tube containing the spleen cells in the aforementioned step to mix the two cells uniformly. The centrifuge tube was placed into a normal temperature centrifuge, and centrifuged at 1,000 rpm for 5 min-10 min.

### Fusion

After the end of the aforementioned cell centrifuging, the supernatant was discarded, the wall of the centrifuge tube was wiped dry with a high-pressure filter paper, and the tube cap was tightened. The precipitated cells at the bottom of the tube were knocked evenly so that the cells were evenly spread on the bottom of the centrifuge tube, and slowly added with 1 ml of polyethylene glycol (PEG), and the centrifuge tube was rotated while addition at 1 ml/min. The centrifuge tube was placed flat on a bench face of a biosafety cabinet to expand the contact area between the PEG and the cells. The centrifuge tube was allowed to stand for 30 s-1 min and added with DMEM preheated in advance, with 1 ml for the first minute, 2 ml for the second minute, 3 ml for the third minute, 4 ml for the fourth minute, and 5 ml for the fifth minute, until the effect of diluting was greater than that with 20 ml of PEG. The fusion was terminated, and the aforementioned mixed cell solution was centrifuged at 1,000 rpm for 5-10 min. The supernatant was discarded, and the pellet was resuspended with the prepared hypoxanthine-aminopterin-thymidine (HAT)-containing culture medium, mixed with the feeder cells prepared in the first step, and plated in a 96-well cell plate at 300 μl/well. The cell growth was observed every day. The culture medium was replaced on the 5-6th day of cell growth, and the cells were detected on the 7-10th day of culture.

### Human-Derived T Cell Acquisition

5 ml of peripheral blood was drawn from a healthy individual, and the peripheral blood was slowly added into 5 to 10 ml of Ficoll (from: GE HealthCare Technologies, Inc.) in an ultra-clean bench to avoid complete mixing of the blood and Ficoll. The mixture was then centrifuged at 800 g for 30 min with the centrifuge set to 5% at acceleration and 0% at deceleration. After the end of the centrifugation, the buffy coat layer was pipetted, and washed with PBS for 1 to 2 times at 600 g for 10 min. The resultant precipitate was placed into in a 24-well plate and cultured using an X-VIVO 15 (from: Lonza Bioscience) medium containing 10% fetal bovine serum and 50 IU/ml recombinant IL-2. The well plate was added with 10 μl of CD3/CD28 activating antibodies (from: STEMCELL Technologies Inc.) for activation, and the cells obtained after 48 h of culture was T cells.

### Construction of CAR-T Cells

The human-derived PDGFRβ CAR virus was obtained by packaging with a second-generation lentiviral packaging system by our research team. The virus was taken at a virus quantity of MOI=20 to infect 1×10⁶ T cells, the cells were centrifuged in a 24-well plate at 600 g for 60 min for infection, and then the T cells were transferred into a fresh culture medium for culture.

### Flow Cytometry

2×10⁵ T (Vector transduced (Vec)-T and PDGFRβ CAR-T) cells were taken, placed into 500 μL of pre-chilled PBS, incubated with a PDGFRβ flow cytometry antibody at room temperature with protection from light for 15 min, washed twice with pre-chilled PBS, and then analyzed with a flow cytometer (Beckman).

### Construction of PDGFRβ-Overexpressing 293T Cells

The human-derived PDGFRβ antigen sequence was found on the Uniprot website and synthesized by Qingke Company. This sequence was then constructed onto a lentiviral vector pCDH-EF1-MCS-T2A-copGFP, with the front and back enzymatic cleavage sites being XbaI and BamHI, respectively. A PDGFRβ antigen lentivirus was constructed using the second-generation lentiviral packaging system, and infected 293T cells in a T25 flask at an MOI=10. Green fluorescent protein (GFP) expression of the cells was detected by flow cytometry 48 h after the infection.

### CAR-T Cell Killing Experiment

The experiment was performed using an intelligent real-time cell monitor (equipment model: CM100-α; manufacturer: Shanghai East China University of Science and Technology; Shanghai Liukecandou Medical Tech Co., Ltd.). 5×10⁵ 293T-PDGFRβ cells were added into an electronic chip well plate, and placed into the machine for monitoring. After 5 h, 5×10⁵ T (Vec-T and PDGFRβ CAR-T) cells were added and the monitoring was continued for up to 18 h.

### Results

### CAR Structure and Construction of CAR-T Cells:

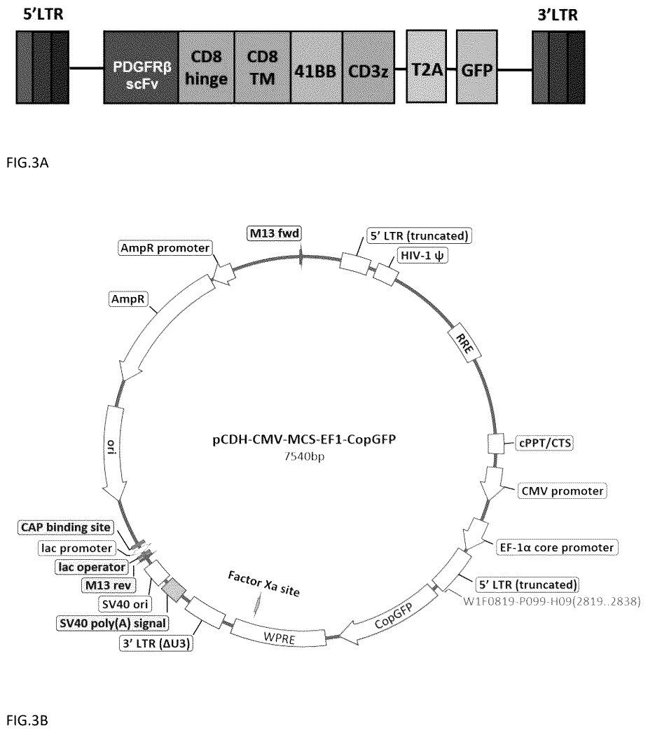

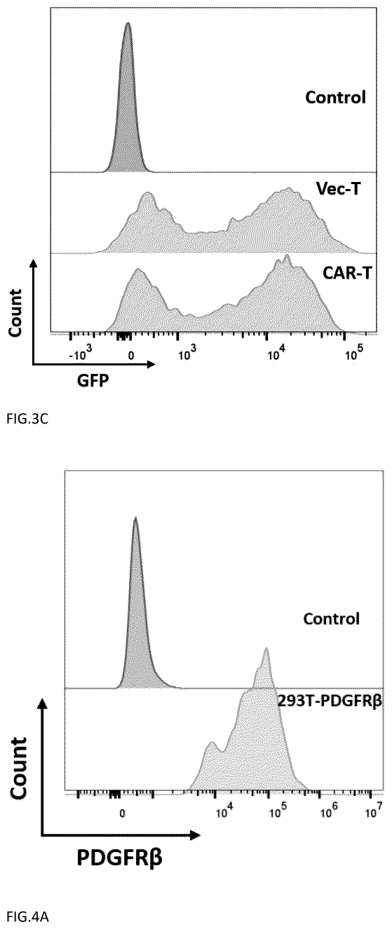

This example used a second-generation CAR with a traditional structure, and used 4-1BB as a costimulatory molecule. The overall CAR structure sequence was CD8 signal peptide-VL-(G₄S)₃-VH-CD8 hinge region-CD8 transmembrane region-4-1BB-CDζ (FIG. 3A). After the CAR structure was synthesized by Qingke Biotechnology Company, we constructed the CAR structure into a lentiviral vector pCDH-EF1-MCS-T2A-copGFP (FIG. 3B), with the front and back enzymatic cleavage sites being XbaI and BamHI, respectively. The sequence was packaged into a lentivirus using a second-generation viral packaging system (with the packaging plasmids of PSPAX2 and PM2G).

T cells were isolated from peripheral blood monocytes of a healthy adult, and then activated with CD3/CD28 activating antibodies (STEMCELL). The T cells were cultured using an X-VIVO 15 medium for infection at an dosage of MOI=20 to obtain CAR-T cells with a high positive rate (FIG. 3C).

The result showed that in the present invention, a second-generation CAR targeting the human PDGFRβ antigen (FIG. 3A) was successfully constructed. By using a lentivirus to infect peripheral blood T cells of healthy human, CAR-T cells with a higher positive rate were obtained (FIG. 3C).

From the aforementioned experiments and results thereof, the following conclusions could be obtained:

A human PDGFRβ CAR was successfully constructed through molecular experiments, and verified by sequencing, and meanwhile human CAR-T cells were successfully constructed, laying the foundation for subsequent CAR-T cell killing experiments.

The Human PDGFRβ CAR-T Cells could Effectively Kill PDGFRβ⁺ Cells In Vitro:

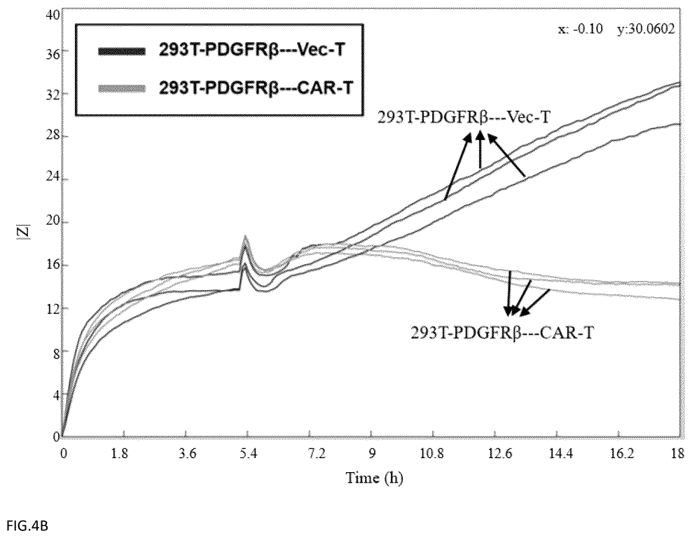

In order to explore the killing ability of the human CAR-T cells to target cells, we constructed human-derived PDGFRβ antigen-overexpressing 293T cells (FIG. 4A), and carried out killing experiments on 293T-PDGFRβ cells with the intelligent real-time cell monitor at an effector-target ratio of 1:1 for a killing time of 18 h.

The result showed that the target cell death rate in the Vec-T cell group was very low, while the target cell death rate in the PDGFRβ CAR-T cell group was relatively high (FIG. 4B).

From the aforementioned experiments and results thereof, the following conclusions could be obtained:

PDGFRβ CAR-T cells had a strong ability to kill PDGFRβ⁺ cells in vitro.

Information about amino acid sequences used in the examples:

ScFv heavy chain sequence:
(SEQ ID NO: 1)
QVQLQQSGPDLVKPGASVRISCKASGYTFTSYYIHWVKQRPGQGLEWIGW
IYPGNVNTKYNEKFKGKATLTADKSSSTAYMQLSSLTSEDSAVYFCARRD
YAINFDYWGQGTILTVSS
ScFv light chain sequence:
(SEQ ID NO: 2)
DIMMTQTPLSLPVSLGDQASISCRSSQSIVHSNGNTYLEWYLQKPGQSPK
LLIYKVSNRFSGVPDRFSGSGSGTDFTLKISRVEAEDLGVYYCFQGSHFP
WTFGGGTKLEIK
G₄S linker sequence:
(SEQ ID NO: 3)
GGGGSGGGGSGGGGS
CD8 signal peptide sequence:
(SEQ ID NO: 4)
MALPVTALLLPLALLLHAARP
CD8 hinge region sequence:
(SEQ ID NO: 5)
TTTPAPRPPTPAPTIASQPLSLRPEACRPAAGGAVHTRGLDFACD
CD8 transmembrane region (TM) sequence:
(SEQ ID NO: 6)
MASPLTRFLSLNLLLLGESIILGSGEA
4-1BB intracellular region sequence:
(SEQ ID NO: 7)
KRGRKKLLYIFKQPFMRPVQTTQEEDGCSCRFPEEEEGGCEL
CD3ζ chain sequence:
(SEQ ID NO: 8)
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR
Amino acid sequence of human PDGFRβ antigen:
(SEQ ID NO: 9)
MRLPGAMPALALKGELLLLSLLLLLEPQISQGLVVTPPGPELVLNVSSTF
VLTCSGSAPVVWERMSQEPPQEMAKAQDGTFSSVLTLTNLTGLDTGEYFC
THNDSRGLETDERKRLYIFVPDPTVGFLPNDAEELFIFLTEITEITIPCR
VTDPQLVVTLHEKKGDVALPVPYDHQRGFSGIFEDRSYICKTTIGDREVD
SDAYYVYRLQVSSINVSVNAVQTVVRQGENITLMCIVIGNEVVNFEWTYP
RKESGRLVEPVTDFLLDMPYHIRSILHIPSAELEDSGTYTCNVTESVNDH
QDEKAINITVVESGYVRLLGEVGTLQFAELHRSRTLQVVFEAYPPPTVLW
FKDNRTLGDSSAGEIALSTRNVSETRYVSELTLVRVKVAEAGHYTMRAFH
EDAEVQLSFQLQINVPVRVLELSESHPDSGEQTVRCRGRGMPQPNIIWSA
CRDLKRCPRELPPTLLGNSSEEESQLETNVTYWEEEQEFEVVSTLRLQHV
DRPLSVRCTLRNAVGQDTQEVIVVPHSLPFKVVVISAILALVVLTIISLI
ILIMLWQKKPRYEIRWKVIESVSSDGHEYIYVDPMQLPYDSTWELPRDQL
VLGRTLGSGAFGQVVEATAHGLSHSQATMKVAVKMLKSTARSSEKQALMS
ELKIMSHLGPHLNVVNLLGACTKGGPIYIITEYCRYGDLVDYLHRNKHTF
LQHHSDKRRPPSAELYSNALPVGLPLPSHVSLTGESDGGYMDMSKDESVD
YVPMLDMKGDVKYADIESSNYMAPYDNYVPSAPERTCRATLINESPVLSY
MDLVGFSYQVANGMEFLASKNCVHRDLAARNVLICEGKLVKICDFGLARD
IMRDSNYISKGSTFLPLKWMAPESIFNSLYTTLSDVWSFGILLWEIFTLG
GTPYPELPMNEQFYNAIKRGYRMAQPAHASDEIYEIMQKCWEEKFEIRPP
FSQLVLLLERLLGEGYKKKYQQVDEEFLRSDHPAILRSQARLPGFHGLRS
PLDTSSVLYTAVQPNEGDNDYIIPLPDPKPEVADEGPLEGSPSLASSTLN
EVNTSSTISCDSPLEPQDEPEPEPQLELQVEPEPELEQLPDSGCPAPRAE
AEDSFL
Amino acid sequence of PDGFRβ antigen extra-
cellular region:
(SEQ ID NO: 10)
LVVTPPGPELVLNVSSTFVLTCSGSAPVVWERMSQEPPQEMAKAQDGTFS
SVLTLTNLTGLDTGEYFCTHNDSRGLETDERKRLYIFVPDPTVGFLPNDA
EELFIFLTEITEITIPCRVTDPQLVVTLHEKKGDVALPVPYDHQRGFSGI
FEDRSYICKTTIGDREVDSDAYYVYRLQVSSINVSVNAVQTVVRQGENIT
LMCIVIGNEVVNFEWTYPRKESGRLVEPVTDFLLDMPYHIRSILHIPSAE
LEDSGTYTCNVTESVNDHQDEKAINITVVESGYVRLLGEVGTLQFAELHR
SRTLQVVFEAYPPPTVLWFKDNRTLGDSSAGEIALSTRNVSETRYVSELT
LVRVKVAEAGHYTMRAFHEDAEVQLSFQLQINVPVRVLELSESHPDSGEQ
TVRCRGRGMPQPNIIWSACRDLKRCPRELPPTLLGNSSEEESQLETNVTY
WEEEQEFEVVSTLRLQHV
Signal peptide sequence:
(SEQ ID NO: 11)
MRLPGAMPALALKGELLLLSLLLLLEPQISQG

The aforementioned examples are only for illustrating the technical concept and features of the present invention, and their purpose is to enable those skilled in the art to understand the content of the present invention and implement it accordingly, but not to limit the claimed scope of the present invention. All equivalent changes or modifications made according to the spiritual essence of the present invention shall be encompassed within the claimed scope of the present invention.

1. A single-chain fragment variable (scFv) targeting human platelet-derived growth factor receptor β (PDGFR-beta or PDGFRβ), comprising a scFv heavy chain V_{H} and a scFv light chain V_{L}; wherein an amino acid sequence of the scFv heavy chain V_{H} is as shown in SEQ ID NO: 1; and an amino acid sequence of the scFv light chain V_{L} is as shown in SEQ ID NO: 2.

2. A chimeric antigen receptor targeting human PDGFRβ, at least comprising the scFv targeting human PDGFRβ according to claim 1.

3. The chimeric antigen receptor targeting human PDGFRβ according to claim 2, wherein:
the chimeric antigen receptor targeting human PDGFRβ is formed by connecting a signal peptide, an antigen binding domain, a hinge region, a transmembrane region, a costimulatory signaling domain and a signaling domain in series;
the signal peptide is a cluster of differentiation 8 (CD8) signal peptide with an amino acid sequence is as shown in SEQ ID NO: 4;
the hinge region is a CD8 hinge region with an amino acid sequence is as shown in SEQ ID NO: 5;
the transmembrane region is a CD8 transmembrane region with an amino acid sequence is as shown in SEQ ID NO: 6;
the costimulatory signaling domain is a 4-1BB costimulatory signaling domain with an amino acid sequence is as shown in SEQ ID NO: 7; and
the signaling domain is a cluster of differentiation 3ζ (CD3ζ) signaling domain with an amino acid sequence is as shown in SEQ ID NO: 8.

4. The chimeric antigen receptor targeting human PDGFRβ according to claim 2, wherein:
the chimeric antigen receptor is formed by connecting a CD8 signal peptide, an antigen binding domain V_{L}-(G₄S)₃-V_{H} that binds to a human PDGFRβ antigen, a CD8 hinge region, a CD8 transmembrane region, a 4-1BB costimulatory signaling domain, and a CD3ζ signaling domain in series.

5. An isolated nucleic acid molecule, encoding the scFv targeting human PDGFRβ according to claim 1.

6. A vector, comprising the nucleic acid molecule according to claim 5.

7. An immunocompetent cell, expressing the scFv according to claim 1; or comprising a nucleic acid molecule encoding the scFv.

8. The immunocompetent cell according to claim 7, wherein the immunocompetent cell is selected from: a T cell, a natural killer (NK) cell, a monocyte, a macrophage, a dendritic cell, and a mast cell.

9. A method for preparing the immunocompetent cell, comprising: infecting a T cell with a lentivirus, wherein the lentivirus is obtained by transfecting a recombinant lentiviral vector into a lentiviral packaging cell, followed by cell culture; and the recombinant lentiviral vector is prepared by inserting a nucleic acid molecule encoding the chimeric antigen receptor according to claim 2 into a lentiviral vector.

10. A pharmaceutical composition, comprising a therapeutically effective amount of the scFv according to claim 1, and a pharmaceutically acceptable carrier.

11. A kit, comprising the scFv according to claim 1.

12. Use of the scFv according to claim 1 in preparation of a product for detecting PDGFRβ expression.

13. An isolated nucleic acid molecule, encoding the chimeric antigen receptor according to claim 2.

14. An immunocompetent cell, expressing the chimeric antigen receptor according to claim 2; or comprising a nucleic acid molecule encoding the chimeric antigen receptor.

15. A pharmaceutical composition, comprising a therapeutically effective amount of the chimeric antigen receptor according to claim 2, and a pharmaceutically acceptable carrier.

16. A pharmaceutical composition, comprising a therapeutically effective amount of the immunocompetent cell according to claim 7, and a pharmaceutically acceptable carrier.

17. A kit, comprising the chimeric antigen receptor according to claim 2.

18. A kit, comprising the immunocompetent cell according to claim 7.
